(19) United States
(12) Patent Application Publication
(10) Pub. No.: US 2012/0308675 A1
(43) Pub. Date: Dec. 6, 2012

(54) COMPOSITIONS FOR REDUCING FAT ON THE HUMAN BODY

(76) Inventor: Dawn Gates, Paisley, FL (US)

(21) Appl. No.: 13/488,396

(22) Filed: Jun. 4, 2012

Related U.S. Application Data
(60) Provisional application No. 61/493,228, filed on Jun. 3, 2011.

(51) Int. Cl. A61K 36/28; A61P 3/00

(52) U.S. Cl. 424/764

(57) ABSTRACT

A composition for reducing fat on the human body includes a mixture of herbs and essential oils. The composition can include a combination of burdock root and chickweed, along with rosemary essential oil. Olive oil may be used to extract the appropriate components from the herbs prior to adding the essential oil. The composition can be applied to the human body to cause a reduction of fat in that area without an increase in fat in other areas.

### CROSS-REFERENCE TO RELATED APPLICATIONS

This application claims the benefit of priority of U.S. provisional patent application No. 61/493,228, filed Jun. 3, 2011, the contents of which are herein incorporated by reference.

### BACKGROUND OF THE INVENTION

The present invention relates to compositions for reducing fat on the human body and, more particularly, to an herb and essential oil composition for reducing fat on the human body as well as reducing total cholesterol LDL in users.

Obesity and excess body fat on individuals is a current problem that not only is a health threat to millions of people, but also puts a heavy strain on healthcare costs. People often want to lose weight, but do not have the motivation, encouragement, time, or the like, to maintain the correct levels of diet and exercise. Many times, a person will start a regime of proper diet and exercise, but, if not seeing results after a short period of time, often get discouraged and discontinue the process.

As can be seen, there is a need for an improved method for reducing fat on the human body.

### SUMMARY OF THE INVENTION

In one aspect of the present invention, a composition for the reduction of body fat comprises burdock root extract; chickweed extract; and rosemary essential oil.

In another aspect of the present invention, a method for reducing body fat on a person comprises applying a composition including burdock root extract, chickweed extract and rosemary essential oil to a location on the person's body.

In a further aspect of the present invention, a composition for reducing body fat prepared by a process comprising heating burdock root and chickweed in oil and maintaining an elevated temperature of a resulting heated mixture for a first predetermined period of time; cooling the heated mixture to room temperature and holding a resulting cooled mixture for a second predetermined period of time; repeating this heating and cooling cycle for at least 5 cycles; filtering off solids to obtain a filtrate; and adding the rosemary essential oil to the resulting filtrate.

These and other features, aspects and advantages of the present invention will become better understood with reference to the following description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a composition for reducing fat on the human body includes a mixture of herbs and essential oils. The composition can include a combination of burdock root and chickweed, along with rosemary essential oil. Olive oil may be used to extract the appropriate components from the herbs prior to adding the essential oil. The composition can be applied to the human body to cause a reduction of fat in that area without an increase in fat in other areas.

The composition of the present invention is formulated to reduce body fat in the area onto which it is applied. While not purporting to advance any particular theory, the composition of the present invention could work by increasing the metabolism and movement of fat to the liver to be processed at the area the composition is applied. The composition can be applied to reduce fat in a given area without increasing fat in other areas.

The composition of the present invention includes herbs, including burdock root and chickweed, and essential oils, such as rosemary essential oil.

The composition, through the extract of the herbs, can include a chemical group called saponins, which can absorb fat from where it is applied and move it to the small intestine where it is excreted.

To make the composition of the present invention, a user would first add herbs to oil and let mixture sit at room temperature (70 degrees) for 1 hour. This can release two chemical families before heat is applied. Next, the mixture is heated with an oil, typically cold pressed virgin olive oil, to an elevated temperature, typically about 125° F. After a predetermined period of time, such as 2 hours, at 125° F., the mixture can be cooled to room temperature for a predetermined period of time, such as at least 2 hours. This heating and cooling cycle can then be repeated, if needed, for a total of at least 2 rotations, typically about 5 rotations. After the cycles are complete, the mixture is filtered to remove the herb solids, typically by passing the mixture through a strainer. The essential oil is then added to the strained liquid, at room temperature.

A user would apply the composition of the present invention to an area where they desire a reduction in body fat The user place a desired amount, typically about a half-dollar sized amount, of composition in their hand and apply on a desired area.

The composition of the present invention may be applied for example, twice a day, to a given area. A female subject applied the composition of the present invention that resulted in a reduction of about 20 inches.

In some embodiments, the composition of the present invention may help, either directly or through the reduction of body fat, control a user's total cholesterol LDL, and triglyceride levels. For example, use has shown that total cholesterol was reduced in 89% of users and LDL was reduced in 92% of users.

It should be understood, of course, that the foregoing relates to exemplary embodiments of the invention and that modifications may be made without departing from the spirit and scope of the invention as set forth in the following claims.

What is claimed is:

1. A composition for the reduction of body fat, the composition comprising:
burdock root extract;
chickweed extract; and
rosemary essential oil.

2. The composition of claim 1, further comprising olive oil adapted for use to extract the burdock root extract from burdock root and to extract the chickweed extract from chickweed.

3. The composition of claim 2, wherein the olive oil is virgin cold pressed olive oil.

4. The composition of claim 1, wherein the composition is prepared by heating burdock root and chickweed in olive oil for a predetermined period of time at about 125° F., cooling to room temperature; filtering off solids to obtain a filtrate;
and adding the rosemary essential oil to the filtrate.

5. A method for reducing body fat on a person, the method comprising:
applying a composition including burdock root extract, chickweed extract and rosemary essential oil to a location on the person's body.

6. The method of claim 5, further comprising preparing the composition by heating burdock root and chickweed in olive oil for a predetermined period of time at about 125° F., and cooling to room temperature; filtering off solids to obtain a filtrate; and adding the rosemary essential oil to the resulting filtrate.

7. A composition for reducing body fat prepared by a process comprising:
heating burdock root and chickweed in oil and maintaining an elevated temperature of a resulting heated mixture for a first predetermined period of time;
cooling the heated mixture to room temperature and holding a resulting cooled mixture for a second predetermined period of time;
filtering off solids to obtain a filtrate; and
adding the rosemary essential oil to the resulting filtrate.

8. The composition of claim 7, wherein the oil is olive oil.

9. The composition of claim 7, wherein the first predetermined period of time and the second predetermined period of time are 2 hours.

10. The composition of claim 7, wherein the elevated temperature is about 125° F.

11. The composition of claim 7, where the process further comprises repeating the heating and cooling steps.
